(19) United States
(12) Patent Application Publication
(10) Pub. No.: US 2023/0161095 A1
(43) Pub. Date: May 25, 2023

(54) UV DISINFECTION OF HIGH-TOUCH SURFACES

(71) Applicant: LUMINATOR HOLDING LP, Plano, TX (US)

(72) Inventors: Werner MALCHEREK, Dachau, Bavaria (DE); Kirk GOINS, Allen, TX (US)

(21) Appl. No.: 17/919,253

(22) PCT Filed: Jun. 5, 2020
(86) PCT No.: PCT/US2020/036261

§ 371 Date: Oct. 15, 2022

Related U.S. Application Data
(60) Provisional application No. 63/023,373, filed on May 12, 2020.

(51) Int. Cl. F21V 8/00; G02B 6/10; A61L 2/10

(52) U.S. Cl. CPC G02B 6/0066; G02B 6/102; A61L 2/10; A61L 2202/11; A61L 2202/121; A61L 2202/15

(57) ABSTRACT

Disinfecting devices (100) can include an electromagnetic wave emitting device (EWE device 110) configured to emit UV radiation (115) and a light guide (120) having a front surface (121). The light guide can be configured to distribute UV radiation received from the EWE device across the front surface and be partially transmissive of visible light. UV radiation received by the light guide and distributed across the front surface can function to disinfect the front surface. The device may be configured such that when positioned between a use device (140) and a user (150), the use device is observable through the disinfection device. Methods for disinfecting may similarly include emitting UV radiation into a light guide, reflecting and diffusing UV radiation within the light guide across the front surface of the light guide thereby disinfecting the front surface; and receiving an interaction with a use device through the disinfection device via the front surface from a user.

### RELATED APPLICATIONS

This application claims the benefit of U.S. Provisional Application 63/023,373, filed May 12, 2020, the contents of which are incorporated herein by reference.

### BACKGROUND

In the public realm, various screens, buttons, and other surfaces may be deemed as high-touch surfaces, because several different individuals may contact them on a daily basis. Disinfection services may only be offered periodically and thus allow multiple users to come in contact with a surface between each cleaning. In such situations, contaminants, such as viruses, may be present on the surface and thus be transferred to from user to user as they come in contact with the surface. In some past examples, UV emitting devices have been used to periodically disinfect such surfaces and the surrounding air. However, such systems do not continuously and efficiently clean such surfaces.

### SUMMARY

Aspects of the present disclosure are directed toward systems and methods for disinfecting a high-touch surface. In some embodiments, a disinfection device includes an electromagnetic wave emitting device (EWE device), the EWE device being configured to emit electromagnetic radiation within the ultra-violet spectrum (UV radiation). A disinfection device may further include a light guide having a front surface and a rear surface. In some configurations, the EWE device may be positioned to emit UV radiation into the light guide, the light guide may be configured to reflect and diffuse a portion of the UV radiation received from the EWE device in a distributed manner across the front surface, and the light guide may be at least partially transmissive of visible light between the front surface and the rear surface. A disinfection device can be further configured such that a use device, positioned such that at least a portion of the disinfection device is between the use device and a user, is observable through the disinfection device via the front surface by the user and physically interactable through the disinfection device via the front surface by the user and the UV radiation received by the light guide and distributed across the front surface functions to disinfect the front surface.

In some embodiments, the disinfection device may comprise a plurality of EWE devices configured to emit UV radiation into the light guide. Additionally or alternatively, the UV radiation received at the front surface is sufficient to remove, denature, or deactivate pathogens located on the front surface.

In some embodiments, the disinfection device may further comprise a UV barrier positioned proximate a surface of the light guide. The UV barrio may be positioned proximate the rear surface of the light guide and be configured to block UV radiation from the EWE device being received by the use device and to be at least partially transmissive of visible light. In some examples, the light guide is configured to reflect and diffuse a portion of the UV radiation received form the EWE device in a distributed manner across the rear surface, and in further embodiments the US barrier is configured to reflect a portion of the UV radiation received at the rear surface of the light guide towards the front surface, whereby the UV radiation distributed across the rear surface is reflected towards the front surface in a distributed manner across the front surface.

In some embodiments, the disinfection device may further comprise an ultrasonic wave generator physically coupled with the light guide such that sonic waves generated by the ultrasonic wave generator are received by the front surface and the sonic waves received by the front surface functions to disinfect the device. In further embodiments, the ultrasonic wave generator may be physically coupled to the rear surface.

In some embodiments, a method of disinfecting a high-touch surface using a disinfection device may comprise emitting UV radiation into a light guide and the light guide having a front surface and a rear surface, and the light guide being at least partially transmissive of visible light between the front surface and the rear surface. A method may further comprise reflecting and diffusing a portion of the UV radiation within the light guide in a distributed manner across the front surface and disinfecting the front surface by the UV radiation reflected and diffused in a distributed manner across the front surface. Additionally, a method may comprise receiving interaction with a use device through the disinfection device via the front surface from a user. In some embodiments, the receiving interaction may include receiving physical contact on the front surface from the user, the physical contact providing the interaction with the use device, the interaction being through the disinfection device, the use device being observable by the user through the disinfection device via the front surface.

In some embodiments, the method may further comprise reflecting and diffusing a portion of the UV radiation within the light guide in a distributed manner across the rear surface and restricting the UV radiation received at the rear surface from being received by the use device by a UV barrier, the UV barrier being at least partially transmissive of visible light. Additionally, the method may further comprise absorbing at least a portion of the UV radiation received at the rear surface via the UV barrier and/or reflecting at least a portion of the UV radiation received at the rear surface towards the front surface via the UV barrier.

In some embodiments, the method may further comprise emitting ultrasonic waves into the light guide, receiving at least a portion of the ultrasonic waves on the front surface, and disinfecting the front surface by the ultrasonic waves received on the front surface. In some examples, emitting ultrasonic waves may further comprise emitting ultrasonic waves via an ultrasonic wave generator physically coupled to the light guide.

### BRIEF DESCRIPTION OF DRAWINGS

The following drawings are illustrative of particular embodiments of the invention and therefore do not limit the scope of the invention. The drawings are not necessarily to scale (unless so state) and are intended for use with the explanations in the following detailed description. Embodiments of the invention will hereinafter be described in conjunction with the appended drawings, wherein like numerals denote like elements.

FIG. 1 illustrates an exemplary embodiment of a disinfection device positioned on a use device.

FIGS. 2A and 2B show exemplary embodiments of a disinfection device positioned on a use device.

FIGS. 3A and 3B provide an exemplary embodiment wherein a disinfection device comprises an ultrasonic wave generator.

FIG. 4A shows an embodiment wherein the use device comprises an actuatable device.

FIG. 4B shows an embodiment wherein the use device comprises a display.

FIG. 5 illustrates a process of adjusting the disinfection device based on the registration of a user.

### DETAILED DESCRIPTION

The following detailed description is exemplary in nature and is not intended to limit the scope, applicability, or configuration of the invention in any way. Rather, the following description provides some practical illustrations for implanting various embodiments of the present invention. Those skilled in the art will recognize that many of the noted examples have a variety of suitable alternatives.

Embodiments described herein generally relate to systems and methods relating to a disinfection device configured to disinfect surfaces using electromagnetic wave emitting devices (EWE devices), such as light-emitting diodes (LEDs), UV radiation creating lamps, ultrasonic wave generators, a combination of such devices, or the like. In some embodiments, the devices may provide radiation or the like to a surface, such as a surface external to a use device. For example, the EWE devices may provide electromagnetic waves to a surface external to a use device.

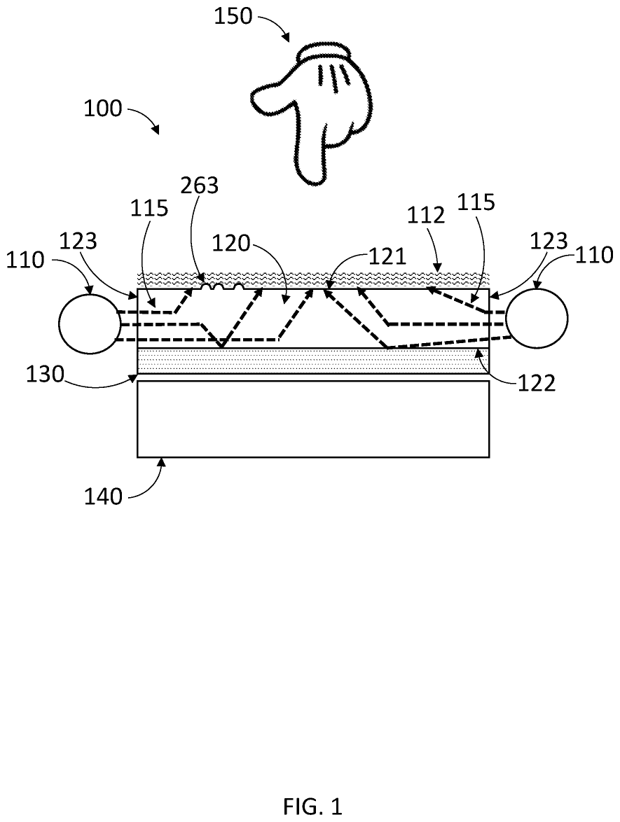

FIG. 1 provides an exemplary embodiment of a disinfection device 100 being positioned on a use device 140. As shown, a disinfection device (e.g. disinfection device 100) may comprise one or more EWE devices 110 as well as a light guide 120. Each EWE device 110 may be configured to emit electromagnetic radiation (e.g. emitted radiation 115) at one or more wavelengths or wavelength bands of interest. In some embodiments, each EWE device 110 may emit electromagnetic radiation in a pattern, such as a cone-shaped pattern. Light guide 120 may comprise front surface 121, a rear surface 122, and an edge surface 123 located between the front surface 121 and the rear surface 122. In some embodiments, the edge surface 123 may be continuous around the light guide, however in other embodiments the edge surface may comprise multiple edge surface portions. The one or more EWE devices 110 may be positioned to emit radiation 115 into the light guide 120. As shown in FIG. 1, the one or more EWE devices 110 may be positioned to emit radiation 115 into edge surface 123 of the light guide, however other embodiments may comprise one or more EWE devices positioned differently, such as to emitting light into other surfaces of the light guide (e.g. the rear surface 122 and/or the front surface 121).

Light guide 120 may additionally be transmissive or partially transmissive of one or more wavelengths or wavelength bands. For example, light guide 120 may be transmissive or partially transmissive of wavelengths emitted by one or more EWE devices (e.g. emitted radiation 115) and/or light within the visible light spectrum. Furthermore, light guide 120 may be configured to reflect and diffuse a portion of the emitted radiation 115 received from the one or more EWE devices 110 in a distributed manner across the front surface 121, the rear surface 122 and/or edge surface 123. Additionally or alternatively, a portion of the radiation emitted from an EWE device may be directly received by front surface 121 and/or rear surface 122, such as by propagating through light guide 120 with no reflections and/or diffusions.

In some embodiments, EWE devices 110 may be configured to emit radiation 115 of wavelengths to remove, denature, kill, deactivate, and/or suppress replication of various pathogens (e.g. pathogens 112) located on or near front surface 121. For example, when disinfection device 100 is configured to remove various viruses and germs (e.g. COVID-19, Influenza, etc.), the EWE devices 110 may be configured to emit pulsed or non-pulsed radiation within the ultraviolet (UV) spectrum, such as electromagnetic radiation with wavelengths between 100 nm and 450 nm. Additionally or alternatively, the EWE devices may be configured to emit other types of radiation, such as radiation outside the UV spectrum (e.g. radiation within the visible light spectrum, infrared spectrum, microwave spectrum, etc.). For example, various pathogens (e.g. bacteria, fungi, parasites or the like) may be more vulnerable to different types of radiation and/or different amounts of radiation. In some embodiments, the EWE devices may emit different types of radiation based on the desired type of disinfection or adjust the radiation emitted based on external factors as discussed herein (e.g. during specific exposure times, on a cycle, based on the amount of emitted radiation in a given timeframe, etc.).

In some aspects of the invention, light guide 120 may be optically coupled to the EWE devices 110 and may be formed form a sheet of glass, plastic, acrylic, or the like. Light guide 120 may be manufactured out of various substrates to provide a bending, reflecting, diffusing, and/or refracting effect on the radiation emitted (e.g. emitted radiation 115) by the EWE devices 110. In some embodiments, light guide 120 may be configured to influence the direction of emitted radiation 115. For example, light guide 120 may direct all or a portion of emitted radiation 115 toward the front surface 121. For instance, light guide 120 may be embedded with diffuser particles that reflect UV light rays as noted elsewhere in this application and suppress total internal reflection (e.g., particularly so if the UV light guide is at least partially transmissive of UV radiation) so as to permit the UV light rays to exit out a top or bottom surface of the UV light rays and contribute to the destruction, reduction, etc. of pathogens on the top surface of the light guide 120. In some embodiments, front surface 121 may comprise a separate substrate, such as a transparent protective layer. Furthermore, light guide 120 may comprise various geometric shapes, such as to fit various use devices 140. For example, light guide 120 can be curved, shaped, bent, rounded, or the like.

Disinfection device 100 may additionally include a UV barrier 130. UV barrier 130 may comprise a separate substrate and/or a coating optically coupled to light guide 120. UV barrier 130 may be located at or near the rear surface 122. Additionally or alternatively, UV barrier 30 may be located on or near the edge surfaces 123, the front surface 121, or the like. UV barrier 130 may comprise various substrates to provide a partial reflectance or total reflectance of radiation emitted by EWE devices 110 in order to protect the use device 140 from the influence of emitted radiation 115. Additionally or alternatively, UV barrier may comprise various substrates to provide a partial absorbance or total absorbance of radiation emitted by EWE devices 110 in order to protect the use device 140 from the influence of emitted radiation 115.

As shown in FIG. 1, the UV barrier 130 is located on or near the rear surface 122 of light guide 120, and radiation received by the rear surface 122 may be reflected towards front surface 121. In such embodiments, the combination of the light guide 120 and UV barrier 130 may help distribute radiation across front surface 121, and in some designs, distribute emitted radiation 115 generally uniformly across front surface 121. For example, light guide 120 may be configured to reflect and diffuse a portion of the emitted radiation 115 received by the one or more EWE devices in a distributed manner across the front surface 121 and the rear surface 122. Furthermore, UV barrier 130 may be configured to reflect emitted radiation 115 received at the rear surface toward the front surface 121, whereby the radiation distributed across the rear surface 122 is reflected toward the front surface 121 in a distributed manner across the front surface 121.

In some embodiments, there may be a desired amount of energy and/or power to be provided to front surface 121, including a sufficient amount of energy and/or power to remove, denature, kill, deactivate, and/or suppress replication of various pathogens to reduce the risk of transmission to a human. Accordingly, EWE devices 110 may emit radiation 115 for a predetermined duration and/or a predetermined intensity to achieve the desired goal. Additionally or alternatively, EWE devices 110 may pulsate emitted radiation 115 on a duty cycle to achieve the desired amount of energy and/or power. In some embodiments, the duration, intensity, duty cycle, or the like of emitted radiation 115 can be adjusted based on the desired amount of energy and/or power to be provided to front surface 121. Furthermore, the amount of energy and/or power provided to front surface 121 may be dependent on the type of radiation emitted and/or external factors discussed herein (e.g. during specific exposure times, on cycle, based on the amount of emitted radiation in a given timeframe, etc.).

As shown in FIG. 1 and described herein, a user 150 may interact with use device 140 via front surface 121. Advantageously, front surface 121 receives radiation (e.g. emitted radiation 115) via a combination of EWE devices 110, light guide 120, and/or UV barrier 130 to provide a disinfected and/or partially disinfected surface which can provide a safer interaction for the user. The amount of disinfection may be dependent on a variety of variables, such as the wavelengths of radiation emitted, the amount of energy (e.g. Watts) of radiation emitted, duty cycle, the length of time the radiation is emitted, and/or the like. For example, if EWE devices 110 emit radiation at a higher energy level and/or higher amplitude, or for a longer duration or a higher duty cycle, more contaminants (e.g. pathogens 112) located on or near front surface 121 may be sterilized, disinfected, and/or deactivated in a given time frame, thus leading to a more efficient and faster percentage of disinfection.

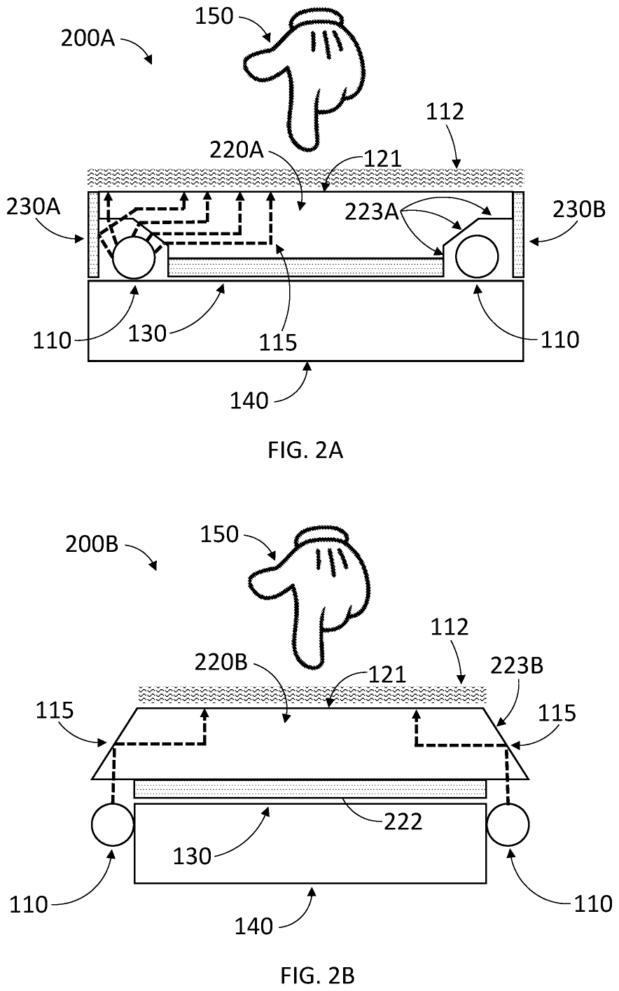

The configuration of disinfection device 100 may depend on the application of the disinfection device and/or the use device. FIGS. 2A and 2B provide alternative embodiments of a disinfection device, such as disinfection device 200A and 200B. FIG. 2A provides an exemplary disinfection device wherein the light guide (e.g. light guide 220A) is shaped to provide a more compact design. As shown, EWE devices 110 are located proximate light guide 220A and are positioned to emit radiation 115 into a plurality of edge surfaces 223. Furthermore, disinfection device 200A comprises UV barriers 230 located on/near edge surfaces 223. UV barriers 230 may be similar to or the same as other UV barriers described herein (e.g. UV barrier 130). As shown in FIG. 2A, UV barriers 230 may be configured to reflect or partially reflect emitted radiation 115 towards light guide 220A and/or towards front surface 121. In some embodiments, the combination of UV barriers (e.g. 130 and 230) and light guide 120 may help distribute radiation across the front surface 121, and in some designs, distribute radiation 115 generally uniformly across front surface 121.

FIG. 2B provides an additional exemplary disinfection device 200B wherein the one or more EWE devices 110 are positioned to emit radiation into a rear surface 222 of light guide 220B. Furthermore, disinfection device 200B may comprise a light guide 220B shaped such that emitted radiation 115 emitted toward edge surface 223B of the light guide 220B is reflected and/or refracted such as to help direct all or a portion of emitted radiation 115 toward the front surface 121. Even though not shown, edge surface 223B may comprise a UV barrier, such as UV barriers 230 or UV barrier 130.

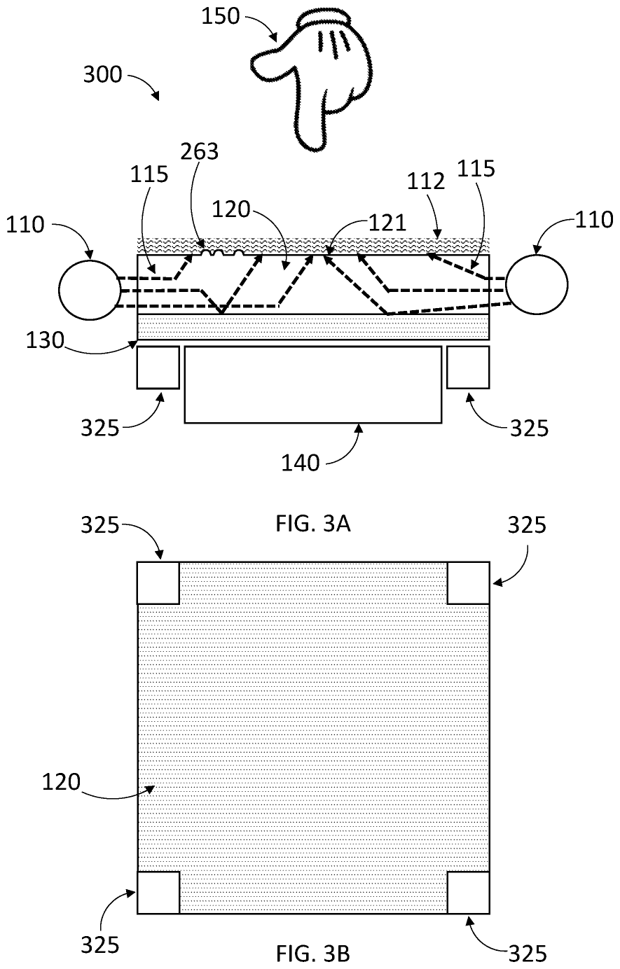

In some embodiments, a disinfection device may comprise additional devices, such as an ultrasonic wave generator. FIG. 3A provides an exemplary embodiment wherein disinfection device 300 comprises one or more ultrasonic wave generators 325. In some embodiments, the one or more ultrasonic wave generator 325 may be positioned to provide ultrasonic waves to front surface 121, such as by being physically coupled to light guide 120 or by emitting ultrasonic waves at the front surface, for instance, when the ultrasonic wave generator 325 is not in direct or indirect physical contact with the front surface 121. As shown in FIGS. 3A 3B, one or more ultrasonic wave generators 325 may be positioned proximate the rear surface 122, such as near the corners of light guide 120. However, other embodiments may comprise one or more ultrasonic generators positioned differently, such as positioned proximate other surfaces of the light guide (e.g. the edge surface 123 and/or the front surface 121).

In some embodiments, ultrasonic wave generator 325 may be configured to emit ultrasonic waves to remove (and potentially also to denature, kill, deactivate, and/or suppress replication of) various pathogens (e.g. pathogens 112) located on or near front surface 121. For example, when disinfection device 300 is configured to remove various viruses and germs, the ultrasonic generators 325 may be configured to emit pulsed or non-pulsed sonic waves, such as sonic waves with wavelengths and frequencies typically associated with ultrasonic waves (e.g., wavelengths less than or equal to about 1.9 cm, frequencies greater than or equal to about 20 kHz).

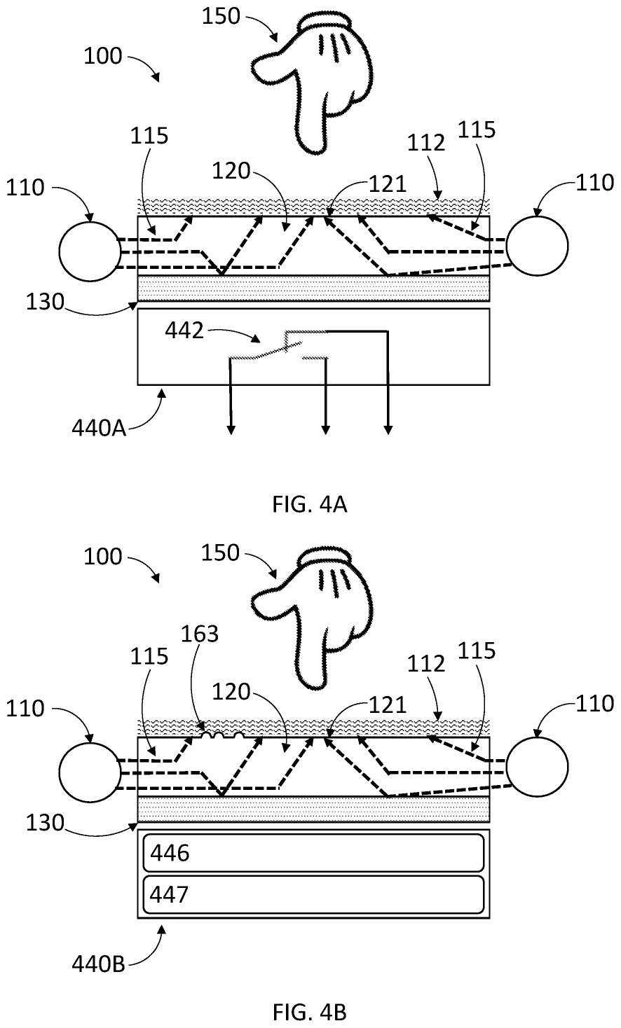

Disinfection devices (e.g. disinfection device 100) can be used in a variety of environments, such as on buttons, switches, displays, fingerprint scanners, windows, doors, or other high touch surfaces. In general, FIGS. 4A and 4B provide two exemplary embodiments comprising the disinfection device 100 and two use devices, use devices 440A and 440B. FIG. 4A provides an exemplary embodiment wherein use device 440A comprises an actuatable device 442. Actuatable device 442 may comprise one or more buttons, switches, keys, sensor surfaces or the like. Exemplary actuatable devices may include stop request buttons, emergency buttons, emergency stop buttons, door open buttons, communication unit buttons on public transportation; elevator buttons, vending machine keys, slot machine buttons, crosswalk buttons, light switches, handles on doors/cabinets, or the like. In such examples, a user may actuate actuatable device 442 (e.g. via pressing, moving, interacting with) via front surface 121 of light guide 120 rather than directly actuating actuatable device 442 of use device 440A. In embodiments wherein use device 440A comprises multiple actuatable devices (e.g. a keypad comprising ten keys for every digit, various floor buttons for an elevator, multiple light switches located adjacent to one another, etc.) each actuatable device may comprise a disinfection device 100. Alternatively, multiple actuatable devices may be covered by a single disinfection device 100.

FIG. 4B provides an additional example wherein use device 440B comprises a display, such as display 447. Display 447 comprise a non-electronic display, such as a map, a public transportation schedule, a sign, printed display, etc. Additionally or alternatively, display 447 may comprise an electronic display, such as an LCD display (e.g. TFT display), LED display, OLED display, ePaper display, or the like. In such embodiments, display 447 may not be compatible with receiving inputs from and/or interacting with a user. However, even though a user (e.g. user 150) may not be able to interact with display 447, a user may still contaminate the surface via coughing, sneezing, breathing, accidentally toughing, or the like. Furthermore, users may still come in contact with display 447, such as by tracing a route with their finger, trying to engage with display 447, etc. Additionally or alternatively, display 447 may comprise attributes for the visually imparted or the like, such as braille (e.g. brail dots 163. Thus, in such embodiments, it may still be beneficial to provide disinfection device 100 on the use device. Additionally or alternatively, display 447 may be configured to be interactable with a user, such as via touch sensor surface 446, a button/switch (e.g. use device 440A), or the like.

In some embodiments discussed herein, a user may interact with a use device (e.g. use device 140, 440A, or 440B). However, continuously disinfecting front surface 121 and/or disinfecting front surface 121 while a user is present may potentially provide discomfort to a user, be against regulations, or the like. In such embodiments, disinfection device 100 may comprise a cycle control mechanism, such that front surface 121 is disinfected after a predetermined amount of time, after a predetermined amount of energy has been provided, at or more predetermined cycles, at one or more predetermined cycles, after a predetermined amount of interactions with a user, and dependent on the efficacy of the particular UV wavelength chosen on the particular pathogens of concern, or the like. Additionally or alternatively, out of an abundance of caution, and to help ease any concerns of the user, it may be beneficial to halt the disinfection process, decrease the intensity of the disinfection processor, or the like while a user is either interacting with the use device (e.g. use device 140, 440A, 440B, or the like) or a sensor, such as a proximity sensor, has detected a user.

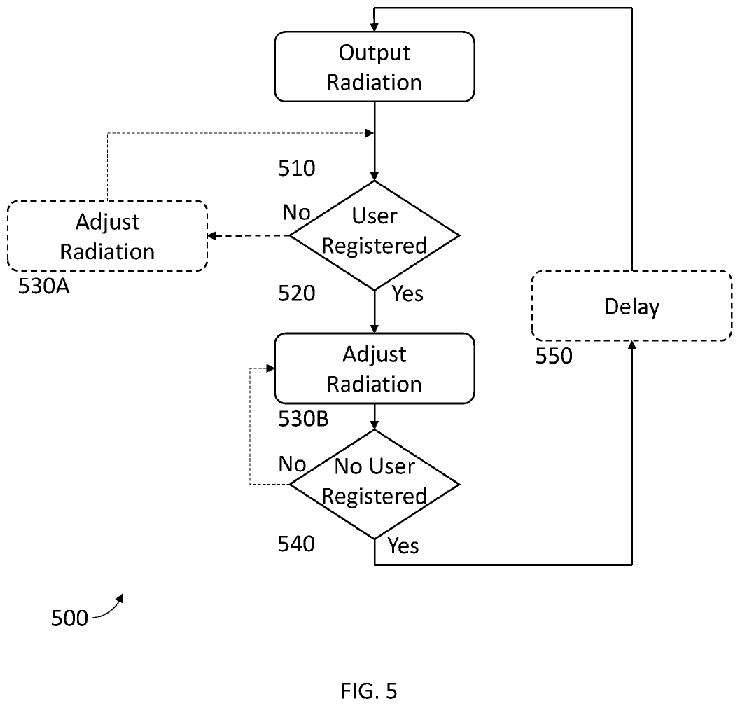

FIG. 5 describes an exemplary embodiment wherein radiation emitted from the EWE devices is adjusted (e.g. not emitted, comprises a different energy level, intensity, duty cycle, wavelength, amplitude, or the like) when the use device is being interacted with by a user or the use device detects the present of a user. Additionally or alternately, as discussed herein, the EWE device may adjust the emitted radiation during other times, such as at times outside of normal operating times for the use device. Initially, the disinfection device (e.g. disinfection device 100) may emit radiation via one or more EWE devices 110 and/or emit ultrasonic waves via one or more ultrasonic wave generators 325 to disinfect the top surface (e.g. front surface 121). As shown, a check may be performed, such as by an external controller or processor, to determine if a user is registered (e.g. Step 520). In some embodiments, if a user is not registered (e.g. No in Step 520), the disinfection device may continue to emit radiation and/or sonic waves. Alternatively, the radiation and/or sonic waves emitted may be adjusted (step 530A). The radiation and/or sonic waves emitted may be adjusted based on a variety of factors, such as the amount of time radiation has been outputted, the amount of energy/power outputted, the amount of energy/power received by front surface 121, the intensity of energy used, the wavelength used, or the like. For example, the EWE device may emit radiation until a predetermined amount of Watts has been emitted and/or the EWE device has been emitting radiation for a predetermined amount of time. Such examples may result in a lower cost of operation by not using EWE devices and/or ultrasonic wave generators unnecessarily.

Once a user is registered (e.g. Yes in Step 520), the disinfection device may adjust the radiation and/or sonic waves emitted (Step 530B), as similarly described with respect to Step 530A. In some embodiments, the amount of radiation and/or the wavelengths/intensity of sonic waves may be reduced or stopped while a user is registered. In such embodiments, the reduction or stoppage may provide a user with a safer or more comfortable experience while using the use device.

A user may be registered in a variety of ways. In embodiments, wherein a user can interact with the use device, a user may be registered based on whether or not an input is received by the use device, an external controller in connection with the use device/disinfection device, or the like. For example, when a button is pressed, a selection is made on a display (e.g. touch sensitive display), or the like. Additionally or alternatively, a user may be registered by an external sensor, such as a proximity sensor, light sensor, motion sensor, etc.

As similarly described check may be performed, such as by an external controller or processor, to determine if no user is present (e.g. Step 540). In some embodiments, if a user is still registered (e.g. No in Step 540), the disinfection device may continue to emit radiation and/or sonic waves (e.g. Step 530B). In some embodiments, the radiation and/or sonic waves emitted may be adjusted again in Step 530B, similar to adjustments described herein. For example, if a user is using a use device for an extended period of time, it may be beneficial to further reduce and/or stop the radiation and/or sonic waves emitted. Such examples may result in a lower cost of operation by not using EWE devices and/or ultrasonic wave generators unnecessarily.

Once a user finishes interacting with the use device, or a user is no longer registered (e.g. the user left the proximity, an external sensor no longer detects the presence of the user, etc.) a user may no longer be registered (e.g. Yes in Step 540). In some embodiments, once a user is no longer registered, the disinfection device (e.g. disinfection device 100) may circle back to outputting radiation to the top surface (e.g. front surface 121). Similar methodologies as discussed with respect to step 520 may be used when determining if a user is no longer registered (step 540). In embodiments wherein a user can interact with the use device, a user may no longer be registered once the user has stopped interacting with the device. For example, once a button is released, a selection has been made, and/or no input has been received for a predetermined amount of time, or the like. Additionally or alternatively, an external sensor or external control unit may be used to determine whether or not a user is present.

In some examples, it may be beneficial to provide a delay (e.g. step 550) between the system no longer registering a user (e.g. step 540) and the disinfection device outputting electromagnetic radiation (step 510). Such a delay may allow a user time to re-register prior to the disinfection device adjusting the emitting radiation upon the top surface to be similar to the radiation emitted prior to a user being registered. Additionally or alternatively, the delay may provide a user time to fully stop interacting with the use device prior to radiation being emitted again.

Disinfection devices as discussed herein (e.g. disinfection device 100) or the like can be used in a variety of situations. For example, a disinfection device may be used in areas with high public contact, such as public transportation stops, governmental buildings, elevators, crosswalks, airports, or the like. Disinfection devices may be used during or after events which increased the risk of disease, such as a concert, sporting event, or the like. Furthermore, disinfection devices may also be used during particular events, such as times when there is a greater risk of transmissible diseases (during or after pandemics, epidemics, bio-terrorist attacks, etc.). For example, disinfection devices (e.g. disinfection device 100) may be used to disinfect surfaces after bio-terrorist attacks, such as the surfaces of military and public transportation vehicles, or the like.

Various embodiments have been described. Such examples are non-limiting, and do not define or limit the scope of the invention in any way.

1. A disinfection device for disinfecting a high-touch surface, comprising:
an electromagnetic wave emitting device (EWE device), the EWE device configured to emit electromagnetic radiation within the ultra-violet spectrum (UV radiation); and
a light guide having a front surface and a rear surface, the EWE device being positioned to emit UV radiation into the light guide, the light guide being configured to reflect and diffuse a portion of the UV radiation received from the EWE device in a distributed manner across the front surface, and the light guide being at least partially transmissive of visible light between the front surface and the rear surface;
wherein a use device, positioned such that at least a portion of the disinfection device is between the use device and a user, is observable through the disinfection device via the front surface by the user and physically interactable through the disinfection device via the front surface by the user; and
the UV radiation received by the light guide and distributed across the front surface functions to disinfect the front surface.

2. (canceled)

3. The disinfection device of claim 2, further comprising a UV barrier positioned proximate a surface of the light guide.

4. The disinfection device of claim 4, wherein:
the UV barrier is positioned proximate the rear surface of the light guide; and
the UV barrier is configured to block UV radiation from the EWE device being received by the use device and to be at least partially transmissive of visible light.

5. The disinfection device of claim 4, wherein the light guide is configured to reflect and diffuse a portion of the UV radiation received from the EWE device in a distributed manner across the rear surface, and the light guide being configured to be at least partially transmissive of visible light between the front surface and the rear surface.

6. The disinfection device of claim 6, wherein the UV barrier is configured to reflect a portion of the UV radiation received at the rear surface of the light guide towards the front surface, whereby the UV radiation distributed across the rear surface is reflected towards the front surface in a distributed manner across the front surface.

7. The disinfection device of claim 4, wherein the EWE device, the light guide, and the UV barrier function to distribute UV radiation received by the light guide generally uniformly across the front surface.

8. The disinfection device of claim 4, wherein the UV barrier comprises a reflective coating located on the rear surface, or a reflective substrate optically connected to the rear surface.

9. (canceled)

10. The disinfection device of claim 4, wherein the UV barrier restricts UV radiation from passing through the UV barrier to reach the use device.

11. (canceled)

12. The disinfection device of claim 1, wherein the use device comprises an actuatable touch surface with visible indicia that invites touching by a user.

13. (canceled)

14. (canceled)

15. (canceled)

16. The disinfection device of claim 1, wherein the EWE device is configured to emit UV radiation into the light guide at a predetermined energy level, and further including a sensor that senses touches to the front surface, the sensor causing the UV radiation emitted by the EWE device to be reduced below the predetermined energy level when the sensor senses touches to the front surface.

17. The disinfection device of claim 16, wherein the sensor is a mechanical sensor when the use device is a depressible button, the mechanical sensor senses depression of the front surface.

18. The disinfection device of claim 16, wherein the disinfection device is configured such that touches to the front surface are electrically communicated to the sensor as indications of touching the front surface, and wherein the sensor is touch sensitive sensor.

19. The disinfection device of claim 16, wherein the EWE device is configured to emit UV radiation into the light guide at the predetermined energy level after a period of time after the sensor stops sensing touches to the front surface.

20. The disinfection device of claim 1, wherein the EWE device is configured to emit UV radiation into the light guide at a predetermined energy level, and further including a sensor that senses objects in close proximity to the front surface, the sensor causing the UV radiation emitted by the EWE device to be reduced below the predetermined energy level when the sensor senses objects in close proximity to the front surface.

21. (canceled)

22. The disinfection device of claim 1, further comprising:
an ultrasonic wave generator physically coupled with the light guide such that sonic waves generated by the ultrasonic wave generator are received by the front surface; and
the sonic waves received by the front surface functions to disinfect the front surface.

23. The disinfection device of claim 22 claim, wherein the ultrasonic wave generator is physically coupled to the rear surface.

24. The disinfection device of claim 1, wherein the UV radiation received at the front surface is sufficient to remove, denature, or deactivate pathogens located on the front surface.

25. The disinfection device of claim 1, wherein the EWE device is configured to emit UV radiation into the rear surface of the light guide.

26. The disinfection device of claim 1, wherein the light guide further has an edge surface located between the front surface and the rear surface, wherein the EWE device is configured to emit UV radiation into the edge surface of the light guide.

27. The disinfection device of claim 26, wherein the EWE device is configured to emit the UV radiation in a cone-shaped pattern, where some of the UV radiation in the cone-shaped pattern received at the edge surface is reflected and diffused by the light guide in a distributed manner across the front surface.

28. (canceled)

29. A method of disinfecting a high-touch surface using a disinfection device, the method comprising:
emitting electromagnetic radiation within the ultra-violet spectrum (UV radiation) into a light guide, the light guide having a front surface and a rear surface, the light guide being at least partially transmissive of visible light between the front surface and the rear surface;
reflecting and diffusing a portion of the UV radiation within the light guide in a distributed manner across the front surface;
disinfecting the front surface by the UV radiation reflected and diffused in a distributed manner across the front surface;
receiving interaction with a use device through the disinfection device via the front surface from a user, the receiving interaction including receiving physical contact on the front surface from the user, the physical contact providing the interaction with the use device, the interaction being through the disinfection device, the use device being observable by the user through the disinfection device via the front surface.

30. The method of claim 29, further comprising:
reflecting and diffusing a portion of the UV radiation within the light guide in a distributed manner across the rear surface; and
restricting the UV radiation received at the rear surface from being received by the use device by a UV barrier, the UV barrier being at least partially transmissive of visible light.

31. The method of claim 30, further comprising absorbing at least a portion of the UV radiation received at the rear surface via the UV barrier.

32. The method of claim 30, further comprising reflecting at least a portion of the UV radiation received at the rear surface towards the front surface via the UV barrier.

33. (canceled)

34. The method of claim 30, further comprising electrically communicating touches from a user one the front surface to the use device as inputs to a touch sensitive display when the use device comprises a touch sensitive display.

35. (canceled)

36. The method of claim 30, wherein emitting electromagnetic radiation further comprises emitting electromagnetic radiation at a predetermined energy level; and further including reducing the electromagnetic radiation below the predetermined energy level when an interaction with the use device by a user is received.

37. The method of claim 36, further comprising emitting electromagnetic radiation at the predetermined energy level after a period of time after an interaction with the use device by the user is no longer received.

38. (canceled)

39. (canceled)

40. The method of claim 30, further comprising:
emitting ultrasonic waves into the light guide;
receiving at least a portion of the ultrasonic waves on the front surface; and
disinfecting the front surface by the ultrasonic waves received on the front surface.

41. The method of claim 40, wherein emitting ultrasonic waves further comprises emitting ultrasonic waves via an ultrasonic wave generator physically coupled to the light guide.

42. The method of claim 30, wherein disinfecting the front surface comprises at least one of remove, denature, kill, deactivate, and/or suppress replication pathogens located on the front surface.

43. The method of claim 30, wherein emitting electromagnetic radiation into the light guide further comprises emitting electromagnetic radiation into an edge surface of the light guide, the edge surface being located between the front surface and the rear surface.

44. The method of claim 43, wherein emitting electromagnetic radiation further comprises emitting electromagnetic radiation in a cone-shaped pattern, where some of the UV radiation in the cone-shaped pattern received at the edge surface is reflected and diffused in a distributed manner across the front surface.

45. The method of claim 44, wherein some of the UV radiation in the cone-shaped pattern received at the edge surface follows a direct path to the front surface.
